(19) United States
(12) Patent Application Publication
(10) Pub. No.: US 2026/0137957 A1
(43) Pub. Date: May 21, 2026

(54) Optimization of Radionuclides for Treatment of Cutaneous Lesions

(71) Applicants: Kenneth Wesley Brooks, Marietta, GA (US); Zachary Gray Carter, Woodstock, GA (US)

(72) Inventors: Kenneth Wesley Brooks, Marietta, GA (US); Zachary Gray Carter, Woodstock, GA (US)

(21) Appl. No.: 18/954,492

(22) Filed: Nov. 20, 2024

(51) Int. Cl. A61N 5/10

(52) U.S. Cl. CPC A61N 5/1029; A61N 5/1071; A61N 2005/1094

(57) ABSTRACT

The present invention provides brachytherapy radiation delivery device designed to topically treat cutaneous skin lesions in patient tissue. The topical radiation delivery device includes a layer of one or more radionuclides. The topical radiation delivery device includes a high Z top shielding layer placed adjacent the side of the radionuclide layer away from the patient tissue. The top shielding layer attenuating the energy of the radionuclide from the environment external to the patient. The patch further includes a bolus layer placed between the patient tissue and the radionuclide layer which attenuates the beta radiation fluence. The one or more radionuclides of the brachytherapy radiation delivery device producing a gamma and beta radiation fluence to achieve a desired Percent Depth Dose within the cutaneous skin lesion.

### CROSS-REFERENCE TO RELATED APPLICATION

This application claims the benefit and is a Continuation in Part of U.S. Utility application Ser. No. 17/503,350 filed on Oct. 17, 2021, and claims the benefit of Provisional Application 63/405,890 filed on Sep. 13, 2022, and claims the benefit of and is a Continuation in Part of U.S. Utility application Ser. No. 17/967,806 filed on Oct. 17, 2022, the entirety of each is incorporated herein by reference.

### REFERENCES CITED OTHER PUBLICATIONS

1. Brauckman et. al. Radiation delivery devices and methods for their manufacture. US Patent Application No. US 2004/0192998A1. Sep. 30, 2004.
2. Conformal therapy is pasted and its Manufacturing approach and use. Chinese patent No. CN106730307A. 2017.
3. Sarazin et al. FABRICATION AND IRRADIATION OF A RADIOACTIVE ISOTOPE SKIN PATCH. US Patent Application No. US 2020/0188691 A1. Jun. 18, 2020.
4. Fischell, et. al. RADIOISOTOPE BANDAGE FOR REDUCING SCAR TISSUE FORMATION. U.S. Pat. No. 6,350,226 B1. Feb. 26, 2002.
5. M. J. Salgueiro, H. Durán, M. Palmieri, R. Pirchio, J. Nicolini, R. Ughetti, M. L. Papparella, G. Casale, M. Zubillaga, Design and bioevaluation of a 32P-patch for brachytherapy of skin diseases, Applied Radiation and Isotopes, Volume 66, Issue 3, 2008, Pages 303-309, ISSN 0969-8043, https://doi.org/10.1016/j.apradiso.2007.09.008
6. Lee J D, Park K K, Lee M G, Kim E H, Rhim K J, Lee J T, Yoo H S, Kim Y M, Park K B, Kim J R. Radionuclide therapy of skin cancers and Bowen's disease using a specially designed skin patch. J Nucl Med. 1997 May; 38(5):697-702. PMID: 9170430.
7. Park et. al. RADIOACTIVE PATCH/FILM AND PROCESS FOR PREPARATION THEREOF. U.S. Pat. No. 5,871,708. Feb. 16, 1999.
8. U.S. Nuclear Regulatory Commission. REGULATORY GUIDE 8.39 REVISION 1: RELEASE OF PATIENTS ADMINISTERED RADIOACTIVE MATERIAL. April, 2020.
9. Dale R G. The application of the linear-quadratic dose-effect equation to fractionated and protracted radiotherapy. Br J Radiol. 1985 June; 58(690):515-28. doi:10.1259/0007-1285-58-690-515. PMID: 4063711.
10. Introduction to Health Physics: Fourth Edition Cember Herman and Johnson Thomas E. McGraw Hill Companies, Inc., New York, NY, 2008. Paperback 864 pp. ISBN: 9780071423083.
11.Definitive and Postoperative Radiation Therapy for Basal and Squamous Cell Cancers of the Skin: Executive Summary of an American Society for Radiation Oncology Clinical Practice Guideline, Likhacheva, Anna et al. Practical Radiation Oncology, Volume 10, Issue 1, 8-20.
12.GEC-ESTRO ACROP recommendations in skin brachytherapy. Guinot, Jose L. et al. Radiotherapy and Oncology, Volume 126, Issue 3, 377-385
13. Khan PhD, Faiz M. and Gibbons, John P. The Physics of Radiation Therapy, 5^{th} edition. Published by Lippincott Williams & Wilkins, 2014. ISBN: 978-1-4511-8245-3
14. “RADIOLOGICAL HEALTH HANDBOOK.” Revised Edition, January 1970. United States Public Health Service. https://doi.org/10.2172/4708654.
   https://www.osti.gov/servlets/purl/4708654.
15. “Live Chart of Nuclides”. International Atomic Energy Agency (IAEA). Vienna International Centre, PO Box 100, A-1400, Vienna, Austria. Web access at https://www-nds.iaea.org/relnsd/vcharthtml/VChartHTML.html.

### BACKGROUND OF THE INVENTION

### Field of the Invention

The present invention relates to the field of external beam radiation therapy and to the field of brachytherapy with localized radiation sources. Particularly, the use of radioactive sources to deliver localized therapeutic radiation to cutaneous lesions of the human or animal body.

### Glossary

ASTRO: American Society for Radiation Oncology

BED: Biologically Effective Dose

Bolus: a slab of material that is placed in the radiation beam usually on the patient's skin which attenuates the beam prior to entry into the patient. This allows the radiation dose and the PDD curve to be shifted towards the surface.

Brachytherapy: use of a radiation source to treat a lesion either adjacently or at a very close distance for a specific amount of time.
a. Contact Therapy: Use of a radioactive device in contact with the patient's skin to treat a cutaneous lesion.

Curie (Ci): A unit of radioactive decay (1 Ci=1000 milliCuries or mCi)

EBRT: External Beam Radiation Therapy, a non-surgical radiation treatment technique where the lesion is treated from a distance by a directed radiation beam that enters through the patient's skin.
a. Electronic EBRT: Use of an electronic radiation-generating machine such as an X-ray tube or linear accelerator to provide EBRT.
b. Radionuclide-based EBRT: use of a radioisotope to provide EBRT.
c. Teletherapy: a historical term that is a synonym of EBRT, usually used to describe EBRT that uses radionuclides such as Co-60 or Cs-137 to produce a radiation beam from a distance of up to 1 meter; these methods were prevalently used by radiation oncologists in the 20^{th} century. Modern teletherapy uses electronic sources from linear accelerators or X-ray tubes to deliver the external beam radiation.

Gray (Gy) or centiGray (cGy): Unit of radiation dose used clinically. (1 Gy=1 Joule per kilogram; 1 Gy=100 cGy).

ISL: Inverse Square Law, the law of physics which states that the intensity of a radiation source reduces in proportion to 1/r\hat{ }2, where r is the distance from the source to the point of measurement.

LINAC: Linear accelerator

NRC: U.S. Nuclear Regulatory Commission

PDD: Percent Depth Dose, the curve generated by dividing the dose at each point along the beam's central axis by the maximum dose on the central axis. Also referred to as “Beam Quality”. A measure of a radiation beam's penetrating power in a medium.

SSD: Source to Surface (or Source to Skin) Distance

Sievert (Sv): Unit of radiation dose used for radiation protection (1 Sv=1000 milli-Sieverts or mSv).

Radioactive isotopes have been used to treat malignant lesions since the early 20^{th} century. In modern times, the term “External Beam Radiation Therapy” (EBRT) expands upon historical isotope-based teletherapy to include high energy photon and electron beams produced by radiation-generating machines such as X-ray tubes and linear accelerators (LINACs).

The market for the treatment of cutaneous skin lesions is now dominated by electronic EBRT devices because of their predictable, on-demand beam delivery in an outpatient clinic setting and dose penetration that can cover deep disease invasion. Despite the greater market share enjoyed by these electronic devices over brachytherapy devices, there are over 3 million patients presenting with non-melanoma skin cancer in the United States each year and there is still an insufficient supply of dermatology-capable radiation therapy machines and dermatology providers who can offer radiation therapy.

Attempts to produce “releasable” brachytherapy devices that can go home with the patient using beta-emitting isotopes have been made to fill the market gap but have failed to become widely adopted by dermatology providers. The clinical failure of these devices can be attributed in each case to the poor choice of the source isotope in the device. The isotopes that have been chosen for other releasable brachytherapy devices all have half-lives and decay energy spectra that are suboptimal, and the designers also failed to optimize the device's geometry and radiation dosimetry for deep dose delivery that penetrates the patient's tissue enough to cover microscopic disease invasion while also keeping the surface dose low enough to not cause permanent damage to skin tissue.

In one example from the prior art (Reference 6), mice were irradiated with an adhesive tape filled with Ho-166. The authors are quoted: “Between 1 and 2 weeks after the completion of Ho-166 therapy, destruction of the tumor tissue was noted, however, acute radiation dermatitis or skin ulceration were developed in all cases,” and “In the treatment of large protruding tumors, beta emitters have a limitation due to their limited penetration range (maximum 8.6 mm). Ninety percent of the energy deposits within 2.1 mm and 10% within 2.1-8.6 mm (11).” The skin ulceration in the mice of this paper may be attributed to the high dose deposition within the first 2 mm combined with the short half-life with a compressed treatment duration. Hence, these results imply that beta-ray based topical radiation patches have two (2) major dosimetric problems that result in them causing more harm to the patient than benefit: (1) skin ulceration and (2) insufficient dose coverage of the tumor. The skin ulceration is attributed to the very high ratio of surface dose (1-2 mm) to deep dose (8-10 mm) and the insufficient tumor dose coverage is attributed to the low dose from beta particles at deep depths (8-10 mm) which does not sufficiently destroy microscopic disease. Hence, these devices do not provide radiation dose that is even close to the ideal dose distribution within the first centimeter of tissue that covers both shallow and deep disease invasion uniformly.

The following paragraphs explain the physical basis of why such beta-ray based brachytherapy devices are a poor choice for contact therapy of cutaneous lesions.

### Source-to-Skin Distance and the Inverse-Square Law

External beam X-ray tubes or LINAC machines typically have a radiation source at some distance between 0 cm and 100 cm from the skin surface. In the case of radioisotope-EBRT, the source angular distribution is isotropic, but in the case of X-ray and LINAC beams the angular distribution of the source may be peaked in one direction but particles are still emitted in all directions. EBRT machines have “collimators” inside their gantry heads which direct the isotropic photon or electron beam into a single collinear direction while shielding out the other directions.

The distance from the source to the treated area's surface is called the Source-to-Surface Distance (SSD, a.k.a. Source-to-Skin Distance). Larger SSDs cause the individual rays of the beam to appear to be collinear with one another. Conversely, smaller SSDs cause the primary rays of the beam to appear to diverge from one another. This effect is a consequence of the Inverse Square Law (ISL) and can also be seen in other contexts such as in the way solar light rays cast linear shadows on Earth's surface even though they are being emitted in all directions radially by the Sun.

### Percent Depth Dose

The key feature of a radiation beam determining its ability to effectively treat the lesion is its “depth-dose” penetration quantified by the “percent depth-dose” (PDD) curve. The PDD curve is a measure of how deeply the radiation can penetrate into the tissue and can be measured in a water-equivalent phantom. The PDD curve is defined as the radiation dose at each point along the central beam axis expressed as a percentage of the maximum dose on the central axis. A “harder” energy spectrum of the incident particles means the particles have a higher energy and therefore penetrate more deeply into the tissue and results in a “high PDD”. Conversely, a “softer” energy spectrum indicates low energy particles and less penetrating beams and therefore a “low PDD”. However, there are additional factors other than the beam energy that affect the PDD. According to Khan's textbook on radiation therapy physics, the three main factors affecting the penetrating power of a photon PDD curve are: (1) Inverse Square Law (2) Exponential Attenuation, and (3) Scattering (see Ref. 13, Chapter 9, section 9.3-C). Importantly, this textbook mentions in section 9.3-C that the PDD decreases rapidly when the source is nearer to the phantom surface which means the penetration of the beam is less when the SSD is small.

### Bolus

“Bolus” is a term well known to those skilled in radiotherapy which refers to the use of a slab of “add-on” material on top of the patient's skin. Usually this material has a water-equivalent density close to 1 g/cc, but any material can act as a bolus. The purpose of the bolus is to cause the first few millimeters of the PDD curve to be deposited into the bolus material instead of the patient, effectively “shifting” the PDD curve to shallower depths in the patient and moving the depth of maximum dose closer to the skin. For example, a 6 MV LINAC photon beam has a 1.5 cm depth of maximum dose in the patient's tissue, but by placing a 1 cm-thick bolus on the patient's skin the clinician can raise the depth of maximum dose to be 0.5 cm instead of 1.5 cm. Thus, one can make small adjustments to the PDD curve within the patient's tissues by using a bolus. The bolus method works for both electron (beta) beams and photon (X-ray or gamma) beams.

### The U.S. Nuclear Regulatory Commission

The U.S. Nuclear Regulatory Commission (NRC) governs the release of radioactive materials into the public and published radiation safety guidelines for release of patients with radioactive materials. The NRC publication NUREG 8.39 REV 1 (see ref. 8) sets the standards for releasability of patients with radioactive materials into the public. Equation (1), (2) and (3) of the NUREG report explain how to estimate the dose to a member of the public for a given released radionuclide. Equations (1)-(3) inform the guidelines in later sections of the report for maximum activity levels allowable for various radionuclides. If a topical radiation patch is designed to be released into the public so the patient does not have to spend the night in the hospital. The topical device must therefore meet the standards for releasability outlined in the NUREG report.

Specifically, the device to be released must not cause a member of the public to receive a dose of more than 5 milliSieverts (mSv), as estimated by equations (1)-(3) of the report. Some radionuclides such as I-131, I-125, and Pd-103 have example calculations and activity limits provided in the NUREG 8.39 report, but many radionuclides do not have pre-calculated releasability criteria. Regardless of whether the releasable activity limit for a given isotope is tabulated in later sections of the report, the isotope to be released must meet the releasability criteria as calculated with equations (1)-(3) of the report at a minimum.

### Biologically Effective Dose and Current Standards of Care in Skin Radiotherapy

The Biologically Effective Dose (BED), dwell time, and fractionation scheme of topical radiation patch brachytherapy should be optimized to provide a therapeutic benefit that matches or exceeds other established radiation treatment regimens in clinical use. Unfortunately, none of the prior art mention methods for calculating the BED of a topical brachytherapy patch. The American Society for Radiation Oncology (ASTRO) publishes Clinical Practice Guidelines for skin cancer radiotherapy (see ref. 11, Table 8). The minimum BED of the published skin cancer treatment regimen in Table 8 is 58 Gray (Gy) and the minimum dose delivered in a single fraction of a given regimen is 180 centiGray (cGy). Consequently, a topical radiation patch designed for radiotherapy treatments should be able to deliver therapeutic dose equivalent to or exceeding the doses and BEDs of other prevalent skin radiotherapy regimens outlined in Table 8 of ref. 11.

### Issues not Addressed by the Prior Art

As will be shown in the next section, examples of prior art have attempted to create a topical radiation patch using beta-radiation (electrons) as the primary treatment particle. Many of these beta-emitting sources do not have a history of use as external teletherapy sources which was the case with high energy gamma emitters such as Co-60, Cs-137 or Radium. The problem with beta emitters that has been overlooked by the prior art is the issue of the ISL causing a shallowing PDD when the SSD is reduced, as discussed in section 9.3-C of Khan's textbook (Ref. 13). Hence, beta-ray emitters such as Ho-166, Y-90 or Re-188 will have reduced penetrating power when they are used as a topical patch compared to when they are used as external teletherapy sources. To provide adequate therapeutic benefit for skin cancer and other cutaneous diseases, the radiation beam should have significant penetration within the first centimeter of tissue. All of the examples of prior art listed in the next section have overlooked the problem of the ISL reducing the PDD, resulting in an inadequate penetrating power and hence poor therapeutic quality for treating cutaneous lesions.

### Summary of the Prior Art

While electronic EBRT machines presently dominate the market for skin treatment, the prior art demonstrates that it may be possible to treat skin lesions with radioisotopes. References 11 and 12 provide a summary table of various approaches to radiation therapy treatment of cutaneous skin lesions, and radioisotope-based approaches are among the techniques outlined therein.

A thin topical radiation patch or film has been proposed by prior inventors. In US20040192998A1 (Brauckman—Ref. 1), a thin film-based patch that can be adhered, sutured, or implanted onto or into flat areas of the body and that can also conform to curved surfaces of the body was proposed. This author states that the film would consist of a substrate containing the isotope Palladium-103. The author specifies that the radioactive Pd-103 would be bonded to the outside surface of the patch through a deposition process. This reference used a low energy gamma emitter Pd-103 which does not have adequate penetrating power for skin therapy, especially when the PDD shallowing effect of the ISL is considered.

In Chinese patent CN106730307A (Ref. 2), the inventors propose a topical radiation patch which treats lesions using beta rays. Reference 2 is quoted as follows: “ray type is pure beta-ray”. The inventors claim that various radionuclides could suffice as the source of beta radiation for beta-ray therapy. The full list of claimed radionuclides is Na-24, K-42, As-67, Y-90, Zr-97, Nb-96, La-140, Ce-143, Sm-153, Ho-166, Re-186, and a combination of Re-188 and Au-198. The inventors quote the beta emission energy of each of these isotopes. In the disclosure of Ref. 2, the radiation patch consists of a filter paper with a water-absorbing resin and a sealing film which surrounds the radioactive paper substrate. Furthermore, the inventors claimed a “dose monitoring alarm” which keeps track of the delivered radiation dosage and sounds an alarm when the treatment reaches the intended dosage. The disadvantage of this device is that it uses “pure beta rays” for the treatment, which do not have adequate penetrating power for treatment of skin lesions topically. A further disadvantage is that the monitoring alarm does not check that physical contact is maintained with the skin at all times during treatment.

In US 2020/0188691 A1 (Sarazin-Ref. 3), the inventors discuss a concept topical patch device which uses lanthanide metal isotopes, phosphorous, or holmium to treat skin lesions. This art is quoted as follows: “β-particles are used instead of γ-rays to provide a more contained radioactive dose”. The disadvantage of this art is that the isotope used is Holmium, which does not produce adequate radiation energy when used topically for treatment of skin lesions. As discussed in [0018] and [0033], the isotope Ho-166 has negative therapeutic effects due to the extreme shallowness of the percent depth-dose curve that causes the majority of the radiation dose to be deposited within the first 1-2 millimeters of tissue. Like the other works of prior art, the inventors of Ref. 6 failed to realize that beta rays alone are insufficient for providing a therapeutic benefit to the patient because of their poor dosimetric characteristics when used for isotope-to-skin contact therapy.

In U.S. Pat. No. 6,350,226 B1 (Fischell—Ref. 4), the inventors describe a radioactive bandage dressing that is intended for use in the reduction of scar tissue. The inventors also claims that their invention utilizes “a beta particle emitting radioisotope as the source of radiation for the radioactive bandage.” The isotope P-32 is mentioned as a possible beta-emitting isotope that could be used because the energy spectrum of P-32 does not allow penetration to depths much deeper than 3.5 mm. This device is only designed to treat surgical incisions for the reduction of scar tissue and wound healing with an elongated strip of radioactive material. The low-energy beta rays and the elongated nature of this device are a disadvantage for curative treatment of cutaneous skin lesions.

In Ref. 5, the inventors designed a P-32 silicone patch for the treatment of skin diseases. Similar to the other referenced prior art, this device also uses a pure beta emitting radiation source which is a disadvantage for treating deep skin cancer invasion.

In Ref. 6, a radioactive patch made from adhesive tape containing Ho-166 was constructed and used to treat skin tumors in mice. As Ho-166 is a beta emitter, this approach is also presented as a pure-beta radiation approach. Notably, the patients developed radiation dermatitis and ulceration at the treatment site, resulting in permanent fibrosis at the treatment site (see [0018] for a

quote from Ref. 6 describing these deleterious effects). This can be attributed to the shallow depth dose deposition of the beta particles as well as the fact that the treatments were not split into multiple fractions which would have allowed the normal tissue more time for repair.

In U.S. Pat. No. 5,871,708A (Park—Ref. 7), a radioactive patch wherein a distributed radiation source is adhered onto a tape and then laminated was proposed. The inventors mention the following: “The radioactive patch/film uses β-rays, differently from previously established method using X-rays, electron rays and γ-rays . . . ”. In this reference, the patch consists of an adhesive tape which contains a stable nuclide that is irradiated with neutrons to produce a radioactive nuclide. Ir-192 is the only high energy gamma emitter that was claimed. The other claimed radionuclides were beta emitters. This art suffers from the same problem as the previously mentioned art, in that the PDD curve produced by any of the claimed isotopes is not adequate for treating cutaneous lesions because of the shallowing effect of the ISL, as well as the low beta energies that are used.

All of the aforementioned prior art claimed the use of beta emitters as the radiation source or claimed beta rays as the particle used for treatment. All of the prior art mentioned above fail to correct for the shallowing effect that the inverse square law has on the PDD curve when using a topical radiation patch. Additionally, the prior art do not provide methods for optimizing the combined fluence of beta and gamma radiation coming from the isotope. In Ref. 6 from the prior art, the mice that were treated with the device experienced erythema, desquamation and ulceration which can be attributed to the very non-uniform dose deposition of the beta rays that were used causing a large beta-ray dose at the skin surface when the dose is prescribed to depths deeper than a few millimeters. These failures of the prior art cause them to have inferior dosimetric properties and poor clinical results when treating cutaneous lesions, and hence this is why these devices have not been placed into widespread clinical use to date.

Beta-ray beams alone are not sufficient to treat cutaneous lesions because they do not exhibit deep penetration to beyond more than 1-2 mm depth when used as a contact therapy device. Hence a device which addresses these issues is lacking in the prior art. It is thus to such an apparatus and method that the present invention is primarily directed.

### SUMMARY OF THE INVENTION

The disadvantages of the prior art are overcome by the present invention which, in one aspect is, a topical radiation delivery device. The device is designed to topically treat cutaneous skin lesions in patient tissue, the device includes a central radioactive layer. The central radioactive layer including at least one uniformly distributed radionuclide and emitting radiation energy in all directions outward. At least one radionuclide sealed within the radioactive layer. A bolus bottom layer is positioned below the central radioactive layer, and a radiation shielding top layer is positioned above the central radioactive layer.

The central radioactive layer including at least one radionuclide emitting energy as gamma particles and beta particles. The at least one radionuclide in the central radioactive layer selected such that the combined energy of the emitted gamma and beta particles results in a desired Percent Depth Dose curve when the topical radiation delivery device is placed upon a cutaneous skin lesion with the bolus bottom layer adjacent the cutaneous skin lesion, for a time period. The molecular concentration of the at least one radionuclide in the central radioactive layer selected to cause the desired fluence of gamma and beta particles to be incident upon the skin lesion and the desired absorbed radiation dose to be deposited into the skin lesion over the time period. The thickness of the bolus bottom layer is selected to attenuate the beta particle fluence at shallow skin depths and an optimal ratio of beta energy to gamma energy is deposited into the skin lesion to achieve a desired fraction of Biologically Effective Dose for the treatment of the cutaneous skin lesion. In other aspects, the topical radiation deliver devise includes at least one radionuclide emitting high energy gamma particles with energy greater than 1 MeV selected to effectively counteract the shallowing effect of the inverse-square law and the Mayneord's effect at short source-to-target distances. When the radiation delivery device is placed upon a cutaneous skin lesion of a patent, the selected activity of a at least one radionuclide and the top layer radiation shielding ensure a member of the public cannot receive a dose of more than 5 milli-Sieverts. An additional removable portable radiation shield is affixed over the topical radiation delivery device, and ensures a member of the public and medical staff receive a dose that is as low as reasonably achievable. In other aspects, the at least one radionuclide includes Na-24, La-140, Ga-66, Y-90, or any combination thereof. The at least one radionuclide is Na-24. The at least one radionuclide emits gamma-photons of energy between 0.5 MeV and 3. The at least one radionuclide includes a gamma-emitter having a physical half-life between 1 hour and 72 hours. In yet another aspect, the topical radiation delivery device is topically affixed over the cutaneous skin lesion using a bandage dressing having a central holder for the radiation delivery device, for the duration of the prescribed time period.

In another aspect, the present invention provides a method to topically treat cutaneous skin lesions in patient tissue. The method including the steps of; Selecting at least one radionuclide, the at least one radionuclide emitting energy as gamma particles and beta particles. Then calculating a required activity of the at least one radionuclide within the central radioactive layer to result in the desired absorbed radiation dose within the targeted lesion. Calculating a required bolus layer thickness to optimize the ratio of beta particle fluence relative to the total combined fluence of beta and gamma particle radiation energy resulting in an optimal Percentage Depth Dose curve within the patient's tissue. Preparing at least one radionuclide with the required radiation activity. Assembling a topical radiation delivery device, the topical radiation delivery device including, a shielding top layer, a bolus bottom layer, and a central radioactive layer. The central radioactive layer including the at least one radionuclide uniformly distributed and emitting radiation energy in all directions outward. The at least one radionuclide sealed within the central radioactive layer. The radiation device having the bolus bottom layer of the calculated thickness between the central radioactive layer and the cutaneous lesion. Then positioning and securing the radiation device over the cutaneous skin lesion. The topical radiation delivery device irradiating the cutaneous skin lesion over a time period. Wherein a desired fraction of Biologically Effective Dose to the skin lesion is achieved. Then removing the topical radiation delivery device from the patient skin.

In other aspects, the method includes a new topical radiation delivery device applied over the cutaneous skin lesion to apply an additional fraction of the Biologically Effective Dose. The step applying a new topical radiation delivery device over the cutaneous skin lesion is repeated until the total Biologically Effective Dose is achieved.

When the topical radiation delivery device is placed upon a cutaneous skin lesion of a patent. the device is substantially radiation sealed external to the patient by the radiation shielding top layer and the patients body. An additional removable portable radiation shield can be affixed over the topical radiation delivery device after positioning and securing the radiation device over the cutaneous skin lesion. This ensures a member of the public and medical staff receive a dose that is as low as reasonably achievable. When the topical radiation delivery device is placed upon a cutaneous skin lesion of a patent, the top layer radiation shielding and the additional removable portable radiation shield ensures a member of the public cannot receive a dose of more than 5 milli-Sieverts.

In other aspects, the at least one radionuclide comprises Na-24, La-140, Ga-66, Y-90, or any combination thereof. The at least one radionuclide comprises Na-24. The at least one radionuclide emits gamma-photons of energy between 0.5 MeV and 3 MeV. The at least one radionuclide is a gamma-emitter having a physical half-life between 1 hour and 72 hours. And the topical radiation delivery device is topically affixed over the cutaneous skin lesion using a bandage dressing having a central holder for the radiation delivery device, for the duration of the prescribed time period.

These and other aspects of the invention will become apparent from the following description of the preferred embodiments taken in conjunction with the following drawings. As would be obvious to one skilled in the art, many variations and modifications of the invention may be affected without departing from the spirit and scope of the novel concepts of the disclosure.

### BRIEF DESCRIPTION OF THE DRAWINGS

FIG. 1 is a vertical cross section of the radiation delivery device depicting the active layer, the bolus layer, the outward-facing layer, the side wall layer, and the patient's skin underneath.

FIG. 2 is a top view of several options for the areal shape of the radioactive patch.

FIG. 3 is a graphical plot of Co-60 teletherapy PDD curves taken from tabulated data in the US Public Health Service Radiological Health Handbook for the 20 cm field size.

FIG. 4 is a plot that includes the same PDD curves from FIG. 3 and two additional curves.

FIG. 5 is a plot of the same curves shown in FIG. 4 with an additional curve depicting the depth-dose distribution from a cylindrical Co-60 source with isotope-to-skin contact calculated by the U.S. NRC code VARSKIN.

FIG. 6 is a plot of the same PDD curves in FIG. 5 with an additional skin-contact PDD curve depicting Na-24 with a 4.75 mm water-equivalent bolus between the source and the skin.

FIG. 7 is a plot of the isotope PDD data from FIG. 6 zoomed in to the first 5 centimeters of tissue.

FIG. 8 is a cross-section of the radiation dose cloud from a 2.3 cm-diameter disk of Na-24 placed upon a phantom and measured with radiosensitive film.

FIG. 9 is a plot depicting a comparison of calculated vs. measured Na-24 PDD curves.

FIG. 11 is a drawing illustrating the shallowing effect of the ISL on the PDD when a fixed radiation source is brought from the teletherapy distance (1) into contact with the surface (2).

FIG. 12 is a scatterplot (1) of all gamma-emitting radioisotopes, plotted as isotope energy versus isotope half-life.

FIG. 13 is a “Dose-Activity Curve”, or DAC (1), used to calculate the required activity to deliver a clinically acceptable therapeutic benefit while remaining within NRC releasability limits.

FIG. 14 is an illustration and mathematical example of the calculation of Biologically Effective Dose from the topical radiation patch treatment using a formula first defined in the British Journal of Radiology (Ref. 9).

FIG. 15 is an illustration and photograph of an experimental measurement of a Percent Depth Dose (PDD) curve from a sealed radionuclide source applied in a contact therapy technique to a water-equivalent phantom.

FIG. 16 is an exploded view of the radiation delivery device.

### DETAILED DESCRIPTION OF THE INVENTION

The present invention provides a safe, releasable and convenient brachytherapy radiation delivery device, also referred to herein as a topical radioisotope patch, or a topical radiation delivery device, with EBRT-equivalent dosimetric and therapeutic properties and is projected to reduce costs to providers and greatly expand access to radiation treatment for patients with cutaneous skin lesions. In order to achieve EBRT-equivalent dosimetry using a radioisotope patch in-contact with the skin, it is necessary to use a source design that includes a mixed source of beta rays and high-energy gamma rays and an optimized bolus layer thickness between the skin and the source which selectively removes a portion of the beta rays out of the beam. None of the prior art have achieved this design to date.

Rationale for using high energy gamma rays: It is likely that prior designs have overlooked the use of high energy gamma rays because high-energy gamma-ray beams have a “buildup region” which accounts for the lower dose in the first few millimeters of tissue wherein the dose rises to the point of maximum dose, after which the dose begins falling again. Gamma photon beams where the beam source is brought close to tissue surface experience a reduced (or shallow) Percent Depth Dose curve caused by a combination of the Inverse Square Law and Mayneord's effect. This phenomenon is described in detail in Ref. 13, Chapter 9, Section 9.3-C. As the source is brought closer to the patient, the point of maximum dose moves closer to the skin surface and the depth of the “buildup” region becomes smaller. If the beam energy is low enough and the source is brought in contact with the skin, the point of maximum dose may actually merge with the skin surface and the buildup region will disappear. If the beam energy is very high, the buildup region may be preserved even when the source is brought in contact with the skin.

Optimal bolus layer depends on the source decay energy: Co-60 is an excellent example of a high energy beam with a buildup region at large SSD of 80 cm but no buildup region when the source is held in contact with the skin. In contrast, Na-24 retains its buildup region even when the source is brought in contact with the skin due to the higher decay energy of the Na-24 gamma photons compared to the Co-60 gamma photons. This makes Na-24 perfect for use in a topical radioisotope patch because the high-energy photons provide deep tissue dose coverage, and the beta-rays that are also emitted by the isotope can be used to fill-in the missing dose in the buildup region. However, as in the prior art the beta rays will cause skin ulceration if the raw source is used without a bolus layer, but when an optimized bolus layer thickness is placed between the source and the skin the beta rays can be selectively attenuated to ensure the optimal beta-ray flux enters the buildup region and provides a uniform therapeutic dose that covers the tumor cells without overdosing the normal tissue and causing ulceration or desquamation. This optimized bolus layer can be used to fine-tune the beta-ray PDD curve but is thin enough that it does not significantly affect the gamma-ray PDD curve. When combined, the two PDD curves produce an optimal dose distribution that covers both shallow and deep tissues with tumoricidal dose without burning the normal skin tissue.

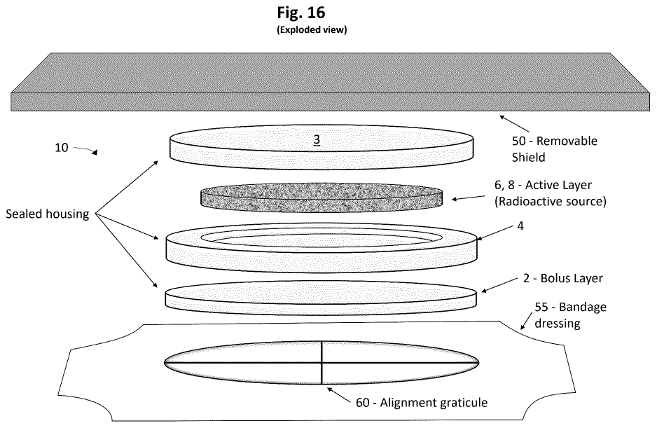

Source housing design: In one embodiment, the present invention provides a sealed housing wherein a radiation source comprising gamma-emitting isotopes, beta-emitting isotopes, or any combination thereof may be stored for use as a topical non-invasive skin treatment patch. The sealed housing consists of (1) the central layer called the “active layer” containing a substrate with a uniform molecular concentration of a decaying radionuclide or combination of decaying radionuclides, (2) an outward facing layer called the “outer layer” which may include a removable radiation shield that can be removed without breaking the seal on the active layer, (3) a sidewall layer called the “sidewall”, (4) and a skin-facing bottom layer also called the “bolus layer” (see FIGS. 1 and 16). The outward facing layer (distal from the patient) with removable radiation shield and the sidewall layer are called the “shielding layers” and are designed to protect members of the public from receiving excessive radiation dose by both providing radiation shielding and by physically sealing the source inside its housing to prevent leakage of source molecules into the environment. The patient's body tissues which are thicker than the removable shield will provide radiation shielding on the skin-facing side with the bolus layer. In other alternative embodiments, the shielding layers may be formed from an appropriate thickness of a high Z material of lead, tungsten, iron, silver, gold, platinum, copper, or brass, or any combination thereof.

Active layer substrate design: Another embodiment of the present invention constructs the active layer such that the radioactivity is distributed uniformly throughout a substrate within the active layer, the active layer comprising the substrate and the radionuclide. As will be appreciated by those skilled in the art, the substate may comprise a carrier which is not radioactive. The effectiveness of the patch depends upon the radioactivity being uniformly distributed throughout the active layer without hot and cold spots of high and low radioactivity. The uniform distribution of radioactivity throughout the central active layer may be achieved by mixing the radioactive molecules with a substrate composed of polymer, gelatinous mixture, liquid, solid, gaseous or other solvent in which the radioactive molecules can be mixed thoroughly and uniformly. In another embodiment, the active layer may comprise the nuclide which is non-radioactive with a portion of the nuclide being radioactive. Such radioactive nuclides are expressed herein as a radionuclide. In one embodiment of the source housing design, the radioactive molecules may be distributed in a powder form throughout the active layer either in raw powder form or in a tablet form containing an excipient binder such as povidone or other binding agent as are known in the pharmaceutical art. As will be appreciated by those skilled in the art, a portion of the molecules, i.e. combination of atoms, within the active layer may be radioactive, and the remaining molecules being non-radioactive. If povidone is used as a binding agent, the povidone may be used in a concentration higher than typical ingestable pharmaceutical drugs because the tablet is not designed to be bioabsorbable or ingested into the patient's body. Hence, a povidone concentration of 5-10% by weight or even up to 50% could be used to ensure the tablet does not flake or release radioactive source molecules into the surrounding environment.

Calculation of absorbed dose from source activity level: In any radioactive isotope, the molecular concentration of the isotope determines the total “activity” (A) of the isotope. Every isotope has a “decay constant” λ which, when multiplied by the numerical molecular concentration N of the isotope gives the activity (A). Mathematically, A=λN. The activity represents the number of decays per second of the source with concentration N and decay constant λ and thus the activity has units of “Decays per second” also known as Becquerels (Bq). Another commonly used unit of activity is the Curie (Ci) which is the number of decays per second that are emitted by 1 gram of Radium-226 (1 Ci=3.7×1010 decays/s=3.7×1010 Bq) and 1000 mCi=1 Ci. In the context of a topical radioisotope patch, the activity is the source's decay rate per second which determines the “fluence” or flux (in particles per cm3) that are emitted by the patch in all directions. Hence, the fluence of particles within the skin tissue is proportional to the activity of the source. Ultimately, the fluence of particles within the skin tissue determines the energy deposited in the tissue. The radiation dose defined as the energy imparted to the tissue in Joules per kg (J/kg). Since 1 Gray (Gy)=1 J/kg, following the chain of reasoning from molecular source concentration to activity, then from activity to fluence and then from fluence to dose, the dose in Gray received by the tissue is ultimately a function of the molecular source concentration in the sealed patch. Practically in a clinical scenario, the provider wants to deliver a specific dose to the lesion. To achieve the goal of delivering the desired dose in Gy to the lesion, the calculation of activity (in mCi) and molecular concentration (in mols or particles) must be performed in reverse starting from the desired dose.

The activity also affects dose to the public: since radiation is emitted in all directions and radiation shielding cannot fully block all emitted rays, an increase in the contained activity in the source will result in an increase in dose to a member of the public as defined by NUREG 8.39 (Ref. 8). Therefore, the prescribed activity must be kept under a limit defined by Ref. 8 which results in a member of the public receiving a dose of less than 5 milliSieverts (mSv). Since a high-energy gamma ray is being used in the source design, the radiation shielding will provide only a limited protection to the public. The more important factor which reduces the public's dose to less than 5 mSv is the selection of an appropriate activity level for the source according the Equation 3 of Ref. 8 (NUREG 8.39 Rev. 1 equation 3). If the activity in mCi is kept below a safe level according to the NUREG 8.39 report, the patch may be considered releasable by regulatory bodies because it will not deliver a dose of 5 mSv or more to a member of the public if released. Hence, the prescribed activity must be chosen by the physician to both (1) achieve a therapeutic gain for the patient and (2) be low enough to be considered safe and releasable by regulatory bodies using guidelines such as NUREG 8.39 Rev. 1. For Na-24, a safe releasable activity limit is initially estimated using equation (3) of Ref. 8 to be 12 mCi.

Source activity and dwell time prescription: In the context of a radioisotope treatment patch for cutaneous lesions, the provider's prescribed dose to the target lesion determines the activity required for the patch. Since the source is constantly decaying over time with half-life T_{1/2}, the activity to achieve the desired dose must be specified at a point in time along with an interval of time in which the integral dose from all emitted particles can be calculated. The most obvious time to specify the prescribed activity is the time of initial patch placement on the skin lesion. This time may be called tₒ. The time at which the patch is removed from the skin may be called t_{f}, assuming the patch remains in contact with the skin between tₒ and t_{f}. The interval of time between tₒ and t_{f} is called the “dwell time,” and it must necessarily be a part of the provider's prescription because it is a factor in determining the delivered dose to the lesion. Shorter dwell times result in fewer particles emitted and hence lower dose, and longer dwell times result in more particles emitted and hence higher dose. Window of time to deliver prescribed dose: The initial prescribed activity A₀ must be specified at a point in time t₀ (such as 9:00 AM on Tuesday). Since the source contains decaying radioisotopes, the activity prior to t₀ will be higher than the desired activity A₀ and the activity at times later than t₀ will be lower than the desired activity A₀. Once sealed, the activity contained in the sealed source cannot be changed. Hence, in order to deliver the exact desired dose D, the physician must place the patch on the patients skin at exactly time t₀. To allow for a margin of error, the dose may be allowed to deviate from the prescribed dose by +/−10%. Hence, a window of time to deliver an acceptable dose to the lesion will be in the interval from time t₋₁ to time t₁ which contains t₀. As an example, in one possible clinical scenario the physician may have between t₋₁=8:45 AM and t₁=9:15 AM on Tuesday to deliver an acceptable dose within +/−10% of the prescribed dose D. If the physician does not place the patch device on the skin between these times, the dose would not be acceptable and this would be considered a mistreatment. The fact that there is a window of opportunity to deliver an acceptable treatment with the radiation patch implies that the source must be made with a higher activity level at the production facility than the final prescribed activity for the patient's treatment. The timing of the source decay between patch production and final treatment delivery is crucial in ensuring that the patient receives the prescribed dose. Hence, externalities which result in patient or provider delays beyond the window of opportunity allowing patch placement will necessitate a re-production of a new patch and retry using the new patch. The initial patch which was not placed within the acceptable window of time must be safely stored for decay in-storage and then discarded.

Possibilities for dwell times: The dwell time could be shorter than 1 half-life of the source, equal to one half-life or longer than 1 half-life. If the dwell time is less than or equal to 10 half-lives of the source, the source will still be radioactive when it is removed. If the dwell time is longer than 10 half-lives, then the source will likely have decayed to a level of radioactivity that is below background radiation and can be safely discarded as long as a survey reading confirms the residual radioactivity levels are below background. If the dwell time is between 0 and 10 half lives, the dose to the target can be calculated and the radioactive device may need to be stored in a shielded container until it has decayed to background levels.

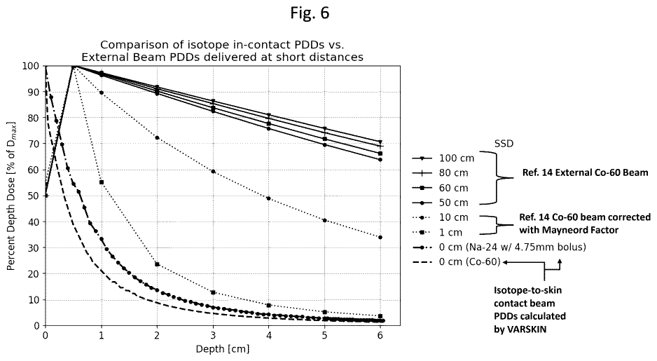

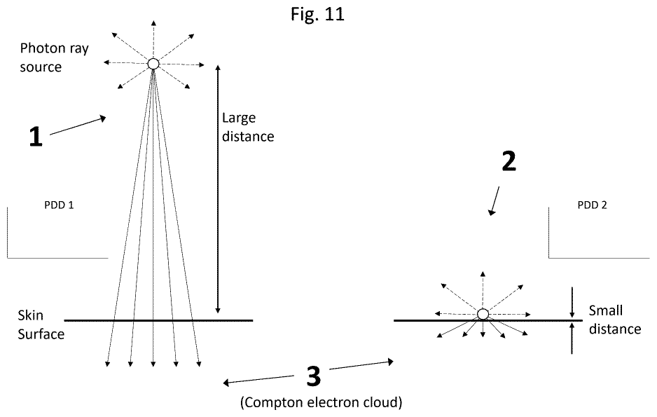

An embodiment of the present invention corrects for the shallowing effect of the Inverse Square Law (ISL) when the radiation source is brought near to the treated surface (see FIG. 11). To correct for the PDD-shallowing effect of the ISL and maintain a penetrating power reasonably equivalent to a teletherapy beam, it is necessary to increase the beam energy and to use gamma photons in addition to beta particles. Isotopes with high-energy gamma emitters in their decay series such as Na-24, which decays to a metastable gamma-emitting state of Mg-24 can fulfill this requirement (see FIGS. 6, 7, 9, and 12).

+

An embodiment of the present invention provides an optimized bolus layer thickness which results in the optimal attenuation of beta particles and gamma particles, giving the optimal ratio of beta fluence to gamma fluence and resulting in the optimal PDD curve for treatment of cutaneous skin lesions. The optimal bolus layer thickness depends upon the specific radionuclide or combination of radionuclides in the active layer.

In another embodiment the present invention provides a portable radionuclide-based external beam radiation therapy (EBRT) device wherein the radionuclide source is brought to a proximal distance within 0 to 5 cm of the patient's skin in order to increase the dose-rate within the tissue, to reduce the required mCi of the radionuclide(s) to provide a therapeutic effect, and to provide a localized radiation beam conformal to the lesion which maximizes radiation fluence within the lesion and minimizes radiation fluence in the surrounding normal tissue. This configuration is referred to as “contact therapy”, being a hybrid modality of external beam radiation therapy and internal brachytherapy.

In another embodiment, the present invention provides a sealed housing as described in the Detailed Description wherein the radioactive isotope contained inside the active layer is Na-24, Ga-66, La-140, Y-90, or Sr-90, or any combination of thereof.

In yet another embodiment the present invention provides a method for accurate placement of the sealed radiation source over a tumor or other cutaneous target. This method involves a bandage or dressing where the sealed radiation source described previously is affixed within the central part of the bandage. The underside of the bandage may have an adhesive layer for affixing the whole bandage to the skin. The sealed source may not move from the central part of the bandage once the sealed source has been placed within the bandage. The bandage has a crosshair guide mark above the central part containing the sealed source. The bandage serves as an adhesive which holds the radiation source in position on the patient's skin over the targeted lesion. The cross-hair marked on the bandage's surface aids the medical technicians to place the bandage and source housing accurately over the disease site.

In yet another embodiment the present invention provides a “contact alarm” for the sealed housing described in the Detailed Description, which creates an audible or visual alarm, and/or an electronic telephone or internet-based notification whenever the bandage or the source housing is removed from contact with the skin. The alarm or notification may be programmed to only be active during the prescribed treatment time of the radiation patch, after such time has passed the alarm may give a final notification to remove the patch and then may automatically turn itself off since the treatment is complete. During the treatment, the alarm may have a display or audible speech stating the remaining time left for completion of the treatment. Since the activity of the radionuclide or radionuclides is known at the time of placement of the patch, the activity and dose at any time during the treatment period may be calculated using the radionuclide's half-life or radionuclides' half-lives.

In another embodiment, the present invention provides an attachable, removable, portable radiation shield which may be affixed to the top side of the source housing (See FIG. 16). The purpose of this removable shield is primarily for the protection of the physician, medical technicians or other radiation workers who will be responsible for the affixing of the patch to the patient's skin. Although the patient may choose to wear the removable shield for their own comfort and protection after leaving the medical facility, the main purpose of the removable shield is to reduce the aggregate radiation dose to the medical staff over the many radiation patch treatments they will be providing. Although the radiation dose from a single radiation patch is low enough to be considered releasable by the NRC guidelines, there is still a concern of aggregate radiation dose to the medical staff who are providing possibly hundreds of radiation patch treatments on an annual basis. Therefore, the removable shield of certain embodiments of the invention serves primarily as a method of reducing radiation dose to medical radiation workers at the treatment facility and secondarily as a source of comfort and additional radiation protection for the patient and their immediate family.

By using the principles presented in the description and illustrated in the Figures, the external beam treatments prevalent in modern cutaneous lesion therapy may be effectively replaced by an optimized brachytherapy patch with equivalent dosimetry that can be worn home by the patient.

### Issues not Adequately Addressed by the Prior Art

The questions that remain unanswered that have caused the devices described by the prior art to remain clinically ineffective and largely unused are as follows:
1. Given a radionuclide-based EBRT source, what happens to the PDD curve when the source is brought from the conventional “teletherapy” distance of 80-100 cm to be adjacent or very near to the treated surface?
2. Given that the Inverse Square Law reduces the penetrating power of the PDD curve as the source is brought near to the surface, does the energy of a topical radiation patch's source need to be increased to maintain similar penetrating power to a teletherapy beam?
3. What Isotopes Are Optimal for Superficial Treatments?
4. What combination of beta (electron) fluence and gamma (photon) fluence results in the optimal PDD curve for treating cutaneous lesions?

### Question 1 Discussion: Compensating for the ISL's Shallowing Effect at Very Short SSD's

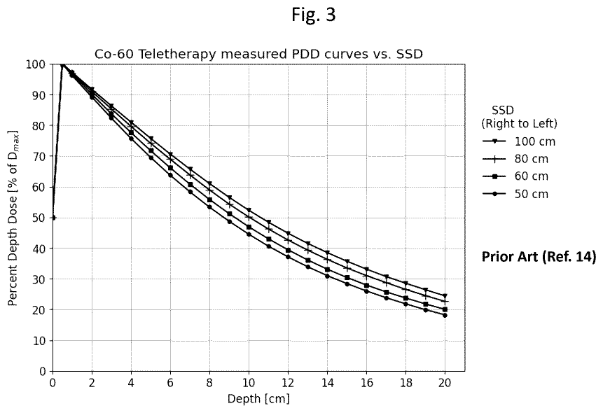

Depicted in FIG. 3 is a graphical plot of Co-60 teletherapy PDD curves taken from tabulated data in the US Public Health Service Radiological Health Handbook for the 20 cm field size. (Original data: Ref. 14, Section III: “Depth Dose Tables”, pgs. 181-182).

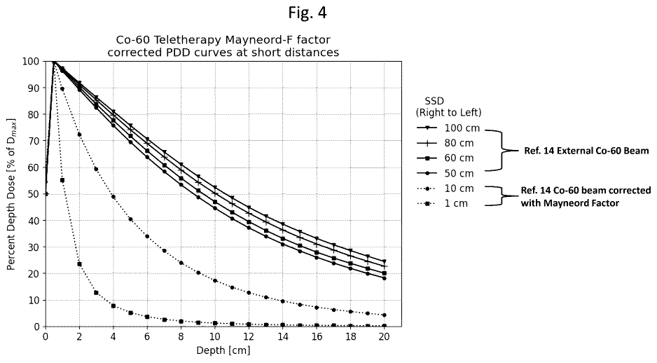

Depicted in FIG. 4 is a plot that includes the same PDD curves from FIG. 3 and two additional curves. To illustrate the shallowing effect of reducing the SSD to very short distances on the PDD relevant to contact therapy, the PDD curve at 80 cm SSD was re-calculated as a PDD curve at 10 cm and 1 cm SSD using Mayneord's F factor. The Mayneord F-factor corrected curves at small SSDs of 1 and 10 cm are depicted in with dotted lines.

As noted previously, there are 3 main factors that determine the Percent Depth Dose curve's characteristics: (1) Source to Surface Distance (SSD), (2) exponential attenuation and (3) scattered radiation. The SSD effect (1) is governed by the Inverse Square Law (ISL) and can be quantified using Mayneord's F factor, as demonstrated in FIG. 4. The exponential attenuation effect (2) is a function of the beam energy spectrum and the medium's density and does not depend upon the distance from the source.

The scattered radiation effect (3) depends upon many factors affecting the Compton Scattering cross-sections at each point in the phantom; the number of Compton-scattered photons in the beam depends upon the field size, the source energy, the phantom density, among other factors. When combined, each of the 3 parameters described above result in the measured PDD curve.

A phenomenon that occurs with Cobalt teletherapy machines and LINACs is that the PDD curve becomes “shallower” as the radiation source is brought closer to skin, i.e. the curve has less penetration at a short SSD than when the source is at a larger SSD (see Ref. 13 Chapter 9.3-C and Ref. 14). This effect is well-explained by the following quote from Khan's textbook: “the drop in dose rate between two points is much greater at smaller distances from the source than at large distances. This means that the PDD, which represents depth dose relative to a reference point, decreases more rapidly near the source than far away from the source” (Ref. 13, Chapter 9, Section 9.3.C). It is important to note that the overall intensity of the radiation fluence is still higher at shorter distances, but the PDD curve is less penetrating because it is a relative comparison of points within the phantom, or human tissue, instead of an absolute intensity at a single point. FIG. 3 illustrates this effect for a Co-60 teletherapy beam by depicting measured PDD curves plotted at SSDs of 50, 60, 80, and 100 cm while holding the field size and energy constant. These curves were created by plotting the tabulated depth-dose data from the US Public Health Service Radiological Health Handbook (see Ref. 14, Section III, “Depth Dose Tables”, pgs 181-182). Note that the PDD data in the reference table begin at 0.5 cm, so the value at 0 cm (surface level) was chosen to be 50% according to the table suggestion in Ref. 14. As may be noted by those skilled in the art, a phantom is an approximation of human tissue used in radiation testing. When using a brachytherapy radiation delivery device the human or animal tissue will experience the same radiation fluence as discussed herein for the phantom.

FIG. 3 illustrates the mild changes to PDD curves that happen over the range of typical external beam teletherapy distances of 50-100 cm. The shallowing effect on the PDD is much more pronounced when the SSD is brought to much shorter depths relevant to brachytherapy of cutaneous lesions. Per section 9.3.C of Ref. 13, Mayneord's F Factor can be used to transform the PDD at one depth and SSD to the PDD at the same depth at a different SSD. Hence, it is possible to approximate PDD curves at shorter SSDs from PDD curves at longer SSDs, and vice versa.

In FIG. 4, the Cobalt-60 PDD curve at 80 cm SSD with a 20 cm 2 field size from FIG. 3 was transformed into a PDD curve at 10 cm and at 1 cm SSD using Mayneord's F factor. It is clear from the 1 cm SSD PDD curve that holding the energy of the beam constant and moving the source to within 1 cm of the surface results in a loss of penetrating power of the Co-60 radiation beam.

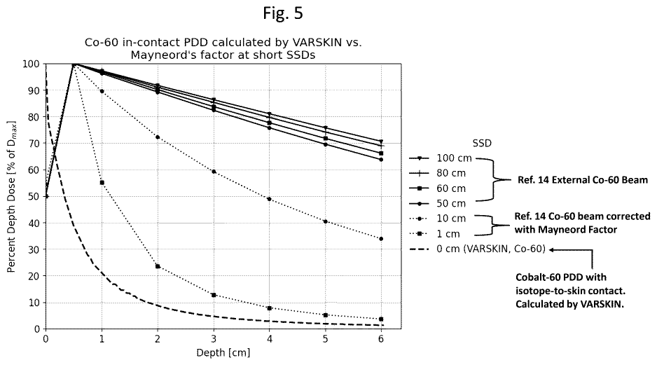

In FIG. 5, to further confirm the SSD vs PDD effect, the US NRC code “VARSKIN” was used to calculate the depth dose curve from a cylindrical Co-60 source capsule, assuming the capsule was left in contact with the skin of a patient. FIG. 5 is a plot of the same curves depicted in FIG. 4 with an additional curve depicting the depth-dose distribution from a cylindrical Co-60 source with isotope-to-skin contact calculated by the U.S. NRC code VARSKIN. The assumptions of the Co-60 VARSKIN source model are: source diameter=2 cm, source thickness=2.5 cm, source density=5.4 g/cc.

In FIG. 5, the curve takes the shape that is predicted by the Mayneord's factor method but is steeper due to being at SSD=0 cm (in contact with skin). The same-energy source of Co-60 at two different SSDs of 0 cm and 80 cm produces radically different PDD curves. As VARSKIN is a well-validated dose computation code based on the Boltzmann transport equation, this serves as a validation of the hypothesis that the ISL greatly reduces the PDD at short distances when the source is kept constant.

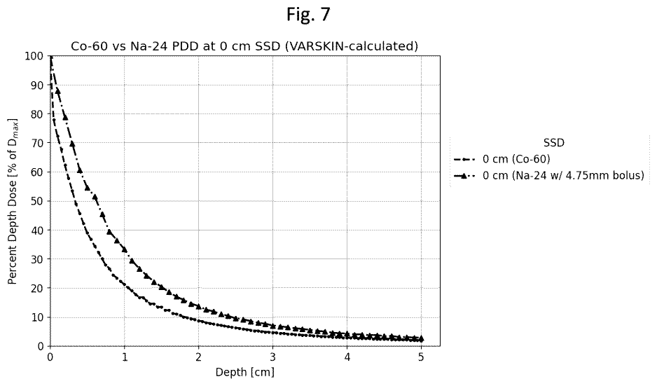

In FIGS. 6 and 7, a model of Na-24 was constructed in VARSKIN with a 4.75 mm bolus layer; the PDD curve for this model is depicted. FIG. 6 is a plot of the same PDD curves in FIG. 5 with an additional skin-contact PDD curve depicting Na-24 with a 4.75 mm water-equivalent bolus between the source and the skin. This means that the isotope is in contact with the bolus and the 4.75 mm thick bolus is sandwiched between the skin and the isotope. FIG. 7 is a plot of the isotope PDD data from FIG. 6 zoomed in to the first 5 centimeters of tissue. This plot illustrates that a Na-24 contact therapy patch is more penetrating than a Co-60 contact therapy patch due to the higher gamma energy of Na-24.

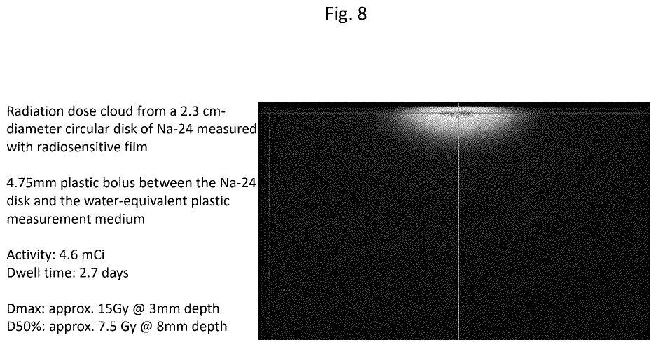

FIG. 8 is a cross-section of the radiation dose cloud from a 2.3 cm-diameter disk of Na-24 placed upon a phantom and measured with radiosensitive film. The thickness of the active layer was 3 mm. The film was sandwiched vertically between two plates of water-equivalent plastic material and the disk and bolus were placed on top of the phantom. There was 4.75 mm of plastic bolus material between the radioactive disk and the plastic phantom to stop the majority of the beta particles from the Na-24. The disk had an initial activity of 4.6 mCi and remained in place for 65 hours.

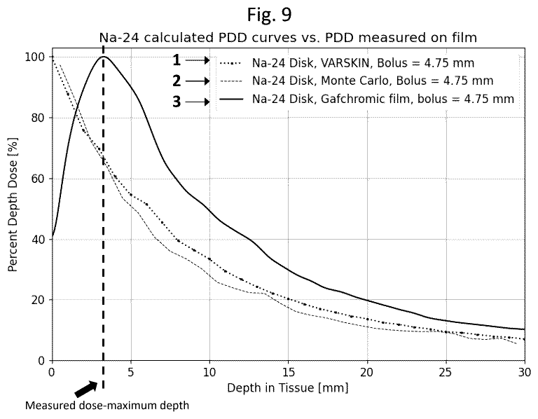

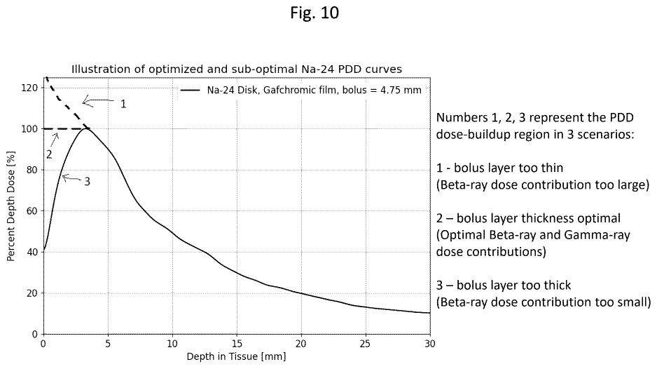

FIG. 9 is a plot depicting a comparison of calculated vs. measured Na-24 PDD curves. Curve (1) is calculated by VARSKIN. Curve (2) is calculated by the Monte Carlo radiation transport code TOPAS-MC. Curve (3) was measured by placing a 2.3 cm disk of Na-24 above a sheet of calibrated radiochromic film with 4.75 mm plastic bolus between the source and the film. The depth of maximum dose measured on the irradiated film is demarcated by a vertical dotted line.

FIG. 10 is an illustration of optimized Na-24 PDD curves and sub-optimal Na-24 PDD curves. Curve (1) illustrates a sub-optimal PDD curve with too much beta-ray fluence at the skin surface caused by a bolus that is too thin. Curve (2) illustrates the optimal Na-24 PDD curve with the right combination of gammas and betas to provide a uniform dose in the gamma buildup region. Curve (3) is the measured Na-24 film PDD curve, which is a sub-optimal PDD curve because the bolus is too thick which removes too many betas from the beam and results in a low dose in the gamma buildup region.

As a conceptual aid, FIG. 11 is an illustration depicting the divergence of the primary rays of the beam when the SSD is brought to near zero. FIG. 11 is a drawing illustrating the shallowing effect of the ISL on the PDD when a fixed radiation source is brought from the teletherapy distance (1) into contact with the surface (2). At a distance, the Compton scattering cross-sections (3) are in approximate alignment with one another, which causes scattered particles to be forward-directed and hence creates a buildup region in the PDD. At the surface, the primary rays of the beam spread out significantly from one another causing lateral spreading of the Compton-scattering cross sections. This effect results in a reduction of the penetrating power of the beam, and hence a shallowing of the PDD curve. The divergence is greater when the SSD is smaller, which causes the Compton-scattered photons to be radially directed instead of forward directed and hence this effect reduces the penetration of the PDD.

### Question 2 Discussion: a High-Energy Gamma Emitter is Needed to Counteract the ISL's Effect

In order to use a topical radiation patch at short “contact therapy” distance while keeping the beam's penetrating power reasonably close to the penetrating power at “teletherapy” distances, the energy of the beam must be increased to counteract the effects of the Inverse Square Law depicted in FIGS. 3-7 & 11. Clearly it is impossible to increase the energy of a radionuclide, so a different radionuclide with a higher energy must be chosen at short distances to compensate for this loss of penetrating power.

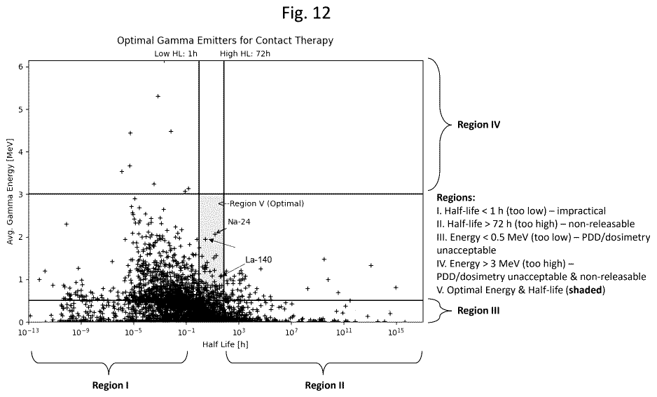

FIG. 12 is a scatterplot (1) of all gamma-emitting radioisotopes, plotted as isotope energy versus isotope half-life. The optimization of radionuclides chosen for a topical radiation patch involves choosing a radionuclide from the “optimal” Region V. This is the region where the half-life is not too low or too high and the energy is not too low or too high. Na-24 is the highest energy-emitting isotope lying within the optimal Region V.

As depicted in FIG. 12, the optimization process of gamma emitters for contact therapy is in choosing an optimal high-energy radionuclide lying within the optimal energy range for contact therapy, but that also has a half-life that is practical for releasable brachytherapy. Beta particles also experience the ISL-effect but they have a finite range and experience significant attenuation in the source material and continuous energy loss in tissue, so beta rays alone do not provide sufficient penetrating power for cutaneous contact therapy. Therefore, a high-energy gamma emitter (which may also be a beta-emitter) will be required for contact therapy. As depicted in FIG. 12, examples of such isotopes that have optimal energy emission for contact therapy which are dual beta/gamma emitters are Sodium-24, Sulphur-38 and Lanthanum-140. As will be appreciated by those skilled in the art, the terms radionuclide and isotope are used interchangeably herein.

### Question 3 Discussion: Optimization of the Radionuclide Choice Considering Half-Life and Energy

A chart of all gamma-emitting isotopes is depicted in FIG. 12. This chart was created from an exhaustive search of the IAEA “Live Chart of Nuclides” database (Ref. 15). A Python code was written to access IAEA's Live Chart of Nuclides API, and the full chart of known nuclides was searched. All isotopes which emit gamma ray photons were included in FIG. 12. Since many gamma emitters are also beta emitters, this chart includes many mixed beta/gamma emitting isotopes. The optimal zone of Half-Life of 1 to 72 hours and energy of 0.5 to 3 MeV is depicted in the shaded Region V. Within the optimal Region V, Na-24 is the highest energy gamma-emitter with an acceptable half-life. Other notable isotopes are Ga-66 and La-140.

For releasable topical brachytherapy of cutaneous lesions, the radiation source must have a short half-life and a high energy. To satisfy the NRC radiation safety requirements the isotope half-life must be short enough that the device will decay to a background level within a reasonable period of less than a few weeks. Hence, Co-60 would not be acceptable as a releasable contact therapy source due to its long half-life of over 5 years. Therefore, a high-energy gamma emitter must be chosen from the set of isotopes that have half-lives within the reasonable range of 1 to 72 hours, as bounded by vertical lines in FIG. 12. A half-life any shorter than 1 hour would be logistically impractical to deliver and a longer half-life than 72 hours would have a decay period causing unacceptable radiation exposure to the public.

### Question 4 Discussion: Optimization of PDD Shape Using Simultaneous Beta and Gamma Radiation

The use of high energy gamma radiation in the 0.5 to 3 MeV range typically results in a “buildup region” in the PDD curve. This buildup region illustrated by FIG. 9(3), FIG. 10(3), and FIG. 11(1), is a result of forward-scattered electrons being released by the primary rays of the beam into the medium. The buildup region ends precisely at the depth of maximum dose where charged particle equilibrium is achieved. For cutaneous lesion therapy, the buildup region is undesirable because it reduces the dose at shallow depths between approximately 0-3 mm deep. An easy way to remove the buildup region is to reduce the gamma energy, but this causes an undesirable shallowing of the PDD curve at deeper depths which is the same problem that was experienced by the prior art with pure beta-rays. The standard of care for cutaneous lesion radiotherapy uses an X-ray machine operating between 50 kVp and 100 kVp. Another machine currently accepted within the standard of care is an accelerator producing a 4-12 MeV electron beam. The present invention provides a Percent Depth Dose (PDD) curve which represents an optimal combination of the PDD's from the two standard of care machines listed above by selectively attenuating the beta particles from the contained radionuclide with an optimal bolus thickness.

Using a mixed beta-ray and photon beam from an emitting isotope will make a more uniform dose distribution that covers both shallow and deep depths. Beta particles have a finite range in tissue after which they completely stop, whereas gamma rays undergo exponential attenuation. Beta particles can be selectively filtered from the emitting isotope beam by placing a selected and precise thickness of bolus material that reduces the beta fluence to a desired level but does not significantly disturb the gamma fluence.

Na-24 is a beta-emitter which also emits gamma ray photons from the metastable states of its decay chain. Hence, a source of Na-24 emits a mixed field of beta-rays and gamma-ray photons. In the development of the present invention, the PDD curve for Na-24 was calculated by the VARSKIN code and by the Monte Carlo radiation transport code TOPAS-MC. The PDD curve of a 2.3 cm Na-24 disk with a bolus thickness of 4.75 mm was also measured using radiochromic film. FIG. 9 shows a comparison of calculated and measured Na-24 PDD curves. The PDD curve calculated by VARSKIN and by TOPAS match one another, but they both fail to match the measured dose on film at shallow depths. Calculation of superficial dose by computer algorithms is notoriously inaccurate, so this result is not surprising and the measured dose on EBT3 film should be taken as the “gold standard”. The measured dose is therefore taken as the gold standard PDD curve to which the calculated PDD curves are compared. It is clear from the measured film dose that the Na-24 disk with 4.75 mm bolus thickness creates a buildup region as described previously.

The term “fluence” is used to describe the intensity of radiation (or number of particles per second) traversing through a given location inside the tissue. “Fluence” is directly proportional to the activity of the source radionuclide which is the number of disintegrations of the radionuclide per second, and activity is directly proportional to the molecular concentration of the source radionuclide. The fluence (or number) of particles at a given point in tissue determines the total energy deposited at that point which is called the “absorbed dose”. In contrast, the energy of the radionuclide only determines how deeply the particles penetrate into the medium which affects the Percent Depth-Dose (PDD) curve.

The combination of beta and gamma particle fluence can be achieved through 2 means. The first method is to combine two radionuclides in the radioactive substrate, one of which is a beta-emitting nuclide and the other a gamma-emitting nuclide. The second method is to use a single gamma-emitting nuclide. Since all gamma-emitting nuclides also emit beta particles as a byproduct of their decay schemes, the emitted isotropic radiation from a gamma-emitting radionuclide always contains fluences of both beta and gamma particles. By adjusting the thickness of the bolus, it is possible to selectively attenuate the fluence of beta particles without significantly disturbing the gamma photon fluence. By optimizing the thickness of the bolus layer, an optimal ratio of beta fluence to gamma fluence can be obtained to achieve desired depth-dose characteristics.

The present invention optimizes the bolus layer's thickness so that the beta particles that make it through the bolus layer are used to “level-out” the buildup region and provide a uniform dose between the surface and the depth of maximum dose. This embodiment of the invention is illustrated by FIG. 10, number (2). The contribution of skin dose from the betas emitted by the isotope can be modulated by changing the thickness of the bolus layer. FIG. 10 shows examples of how the resulting PDD curve looks when the bolus is too thin (1), just right (2), and too thick (3). In FIG. 10 case (1), the dose contribution from betas is too high because too many betas escape the bolus. In the case of (3), the dose contribution from betas is too low because the betas are completely stopped in the bolus and do not reach the skin. In case (2) the contributions from betas and gammas in the buildup region is balanced and provides a uniform dose, which is achieved by making the bolus layer thickness “just right”. Of note in the present invention, the beta particles are used to add superficial dose to compensate for the gammas'low-dose buildup region. The photon PDD curve beyond the depth of maximum dose is preserved because the betas have a finite range and do not affect the photon/gamma dose at deep depths. FIG. 10 illustrates this principle and shows examples of suboptimal and optimal PDD curves from a mixed gamma and beta-ray source. The optimal curve in FIG. 10(2) is the curve which provides a uniform dose in the gamma buildup region but does not disturb the gamma fluence beyond the buildup region.

As will be appreciated by those skilled in the art, the gamma-emitting and/or beta-emitting isotopes'radiation decay spectra may also contain other clinically insignificant particles such as neutrinos or characteristic X-rays as these are immaterial to the therapeutic effect of the source.

### Device Construction and Geometry

Generally stated and as depicted in FIG. 1, the present invention is a sealed radiation delivery device 10 to be used in a topical application upon the skin of a human or animal. The sealed radiation delivery device 10 comprising at least a sealed housing assembly containing a central cavity 1 that holds a radioactive isotope 6. FIG. 1 is a cross section of the sealed radiation delivery device 10. The sealed housing assembly is configured to be placed upon a patient's skin 5 over a skin lesion. The sealed housing assembly comprises an outward facing layer 3, sidewall layers 4, and a skin-facing layer 2. The skin-facing layer 2 is called the “bolus” or “bolus layer”. The sealed radiation delivery device 10 includes the outward facing layer 3, side wall layers 4, skin-facing layer 2, and the radioactive isotope 6 position within the central cavity 1. The radioactive isotope 6 within the sealed housing's central cavity 1 is called the “active layer.”

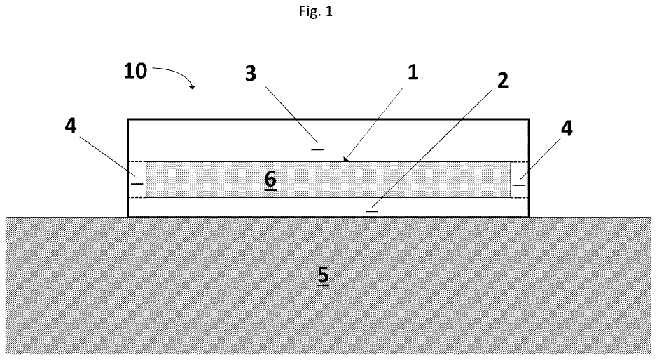

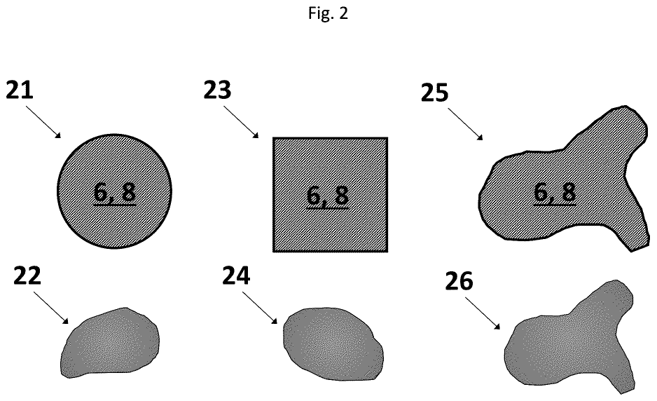

The areal central cavity 1 shape perpendicular to the widest dimensions of the device, and perpendicular to the patient's skin 5, may be one of cylindrical, square, rectangular, or custom to fit the shape of a specific lesion. The radioactive isotope 6 may be integral with the central cavity 1, or formed into a radioactive patch 8 via a substrate and placed within the central cavity 1. FIG. 2 is a top view of several options for the areal radioactive patch 8 shape of the radiation delivery device 10, depicting circular 21 and square 23 patch 8 designs for relatively simple lesion shapes 22, 24 and a custom 25 patch 8 design for a complex-shaped lesion 26. The radioactive isotope 6 residing in the central cavity 1 may be incorporated into a substrate such as a gel, polymer, saline, powder, metallic, or resin-based material to form a radioactive patch 8, or may instead reside within the central cavity 1 in its natural or elemental form, or both.

### Optimized Bolus Layer Thickness

Furthermore, a radiation delivery device embodying features of the present invention when containing a gamma-emitting and beta-emitting nuclide has a bolus layer with a carefully chosen physical thickness which results in an optimal ratio of beta-particle fluence to photon-particle fluence at the skin-facing side of the bolus layer. The optimal bolus layer thickness provides a uniform dose within the gamma buildup region by allowing some of the emitted beta particles to pass through and contribute dose to the gamma buildup region. The optimal thickness of the bolus layer may be determined by measuring the percent depth-dose curve in water or a similar material beneath the skin-facing side of the bolus with varying bolus layer thicknesses and with a fixed number of milliCuries of the radiation source during each test and choosing the thickness which provides the most uniform dose within the first 0 to 4 millimeters of the PDD. A bolus material and thickness is evaluated and selected for each radiation source and PDD desired. In alternative embodiments of the present invention, multiple gamma-emitting radionuclides, beta-emitting radionuclides or gamma-emitting radionuclides and beta-emitting radionuclides may be present in the active layer.

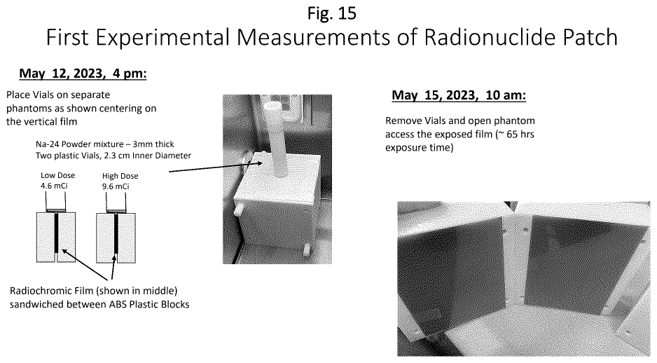

FIG. 15 is an illustration and photograph of an experimental measurement of a Percent Depth Dose (PDD) curve from a sealed radionuclide source applied in a contact therapy technique to a water-equivalent phantom. The measurement consisted of a 2.3 cm diameter, 3 mm thick disk containing a uniform density of Na-24 placed atop a plastic phantom containing Gafchromic™ EBT3 film perpendicular to and centered underneath the disk. This experiment allows for the direct measurement of the PDD curve as well as the 2D symmetric radiation dose distribution beneath the source.

An example of an experimental procedure for measuring the PDD of the radiation patch is depicted in FIGS. 8 and 15. In this experiment, a 2.3 cm diameter, 3 mm thick patch of Na-24 with a bolus layer of 4.75 mm plastic was used to irradiate Gafchromic™ EBT 3 radiation film. This measurement setup allows for both (1) PDD along central axis measurement and (2) viewing and analysis of isodose distribution along the central 2D plane perpendicular to the source housing. To determine the optimal bolus layer thickness, multiple sequences of this experiment must be run with varying bolus layer thicknesses. The optimal bolus layer thickness will be the bolus layer thickness that results in a PDD curve that most closely resembles FIG. 10, No. 2.

### High Energy Photons Counteracting the Shallow PDD Caused by the Inverse-Square Law

A radiation delivery device embodying features of the present invention contains a central radioactive layer, and this active layer emits radiation in all directions outward from the active layer. A result of the inverse-square law is that the depth-dose curve in the patient's tissue beneath the bolus layer is less penetrating when the device is placed adjacent to the skin when compared to the PDD curve from the same radiation with the device at a greater distance. Therefore, a device embodying features of the present invention counteracts the inverse-square law's effect on the PDD when the device is near to the skin. One method of counteracting this effect is for the device to use a gamma-emitting radionuclide such as Na-24, La-140, or Ga-66 with a high energy decay scheme that emits photons in the range of 0.5 MeV to 3 MeV. The higher energy of these photons has the effect of increasing the penetration of the PDD curve into the tissue to cover deep lesion invasion, effectively counteracting the shallowing effect of the inverse-square law at short source-to-target distances.

### Radiation Source having an Optimized Half-Life and Optimized Radioactivity Level

A radiation delivery device embodying features of the present invention, having an active layer as described above, may be used in an outpatient setting where the device is affixed to the patient's body and the patient is allowed to return home with the device. In this treatment setting, the source activity may not exceed an amount that causes the dose to a member of the public to exceed regulatory levels set by the United States Nuclear Regulatory Commission (NRC). In order to satisfy these regulatory limits, the source must have a relatively short half life, since long-lived radionuclides are not considered releasable by the NRC. A source with a half life between 1 hour and 72 hours should satisfy the half-life requirement, hence a device embodying features of the present invention should use radionuclides with physical half-lives within this range and is therefore said to have an “optimized” half-life.

The device may also be used in an outpatient or inpatient clinic setting where the patient remains in the clinic for the entire duration of the treatment. In this treatment scenario, the radioactivity level in the device is not required to meet NRC-NUREG 8.39 releasability guidelines. In such scenarios, the clinician may desire to give a higher dose radioactivity level and/or to reduce the total treatment time which requires using a higher amount of radioactivity to achieve the same therapeutic effect.

A device embodying features of the present invention with a given radionuclide in the active layer when used in an outpatient setting where the device is released into the public should have an activity level that results in the dose to a member of the public receiving less than or equal to 5 milliSieverts (mSv), as can be calculated following the formalism of the NUREG 8.39 guidelines'equations (1), (2) and (3) as published by the NRC (see ref. 8). In contrast to the upper limit on activity, the device must also have a minimum radioactivity level to provide a therapeutic benefit to the patient. The minimum radioactivity level to provide a therapeutic benefit must be determined by the Authorized User and the physician in charge of the patient's treatment following accepted standards of practice and treatment parameters, including but not limited to the Biologically Effective Dose (BED) and historical data on clinical outcomes. Hence, a device embodying such features of the present invention which provides a therapeutic benefit to the patient without exceeding reasonable radiation safety limits is said to have an “optimized” radioactivity level.

### Calculation of the Minimum Activity to Provide a Therapeutic Benefit

A device embodying features of the present invention must provide a therapeutic benefit to the patient being treated. To calculate the minimum radioactivity required to deliver a therapeutic benefit, the user of such a device may use the “Biologically Effective Dose” (BED), a concept well known to those skilled in the art of radiotherapy. A graph called the “Dose-Activity Curve” may be designed using the BED concept which displays the physical dose (Gy), the activity (mCi), the BED (Gy), and the number of treatment fractions (n) all on the same plot. The BED is calculated using the well-known result from Roger Dale; see ref. 9, equations (5) and (F). The physical (given) dose is calculated using a classical result from radiation physics called the “Committed Dose” equation; see ref. 10 for a derivation of this result.

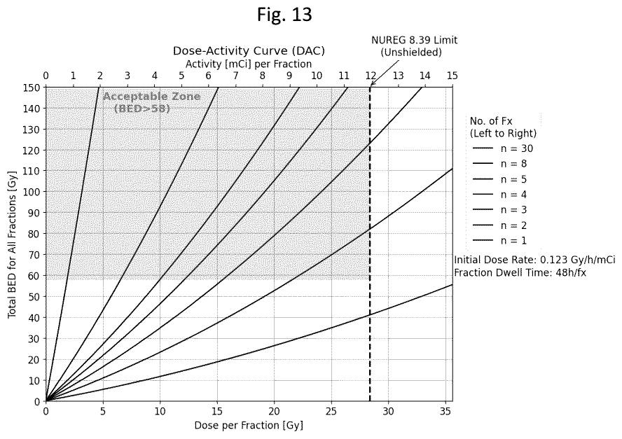

An example of the Dose-Activity curve is depicted in FIG. 13. FIG. 13 is a “Dose-Activity Curve”, or DAC (1), used to calculate the required activity to deliver a clinically acceptable therapeutic benefit while remaining within NRC releasability limits. The acceptable therapeutic zone is bounded by the minimum BED level of 58 Gy published by ASTRO (Ref. 11, Table 8) and a releasable activity limit calculated with equation 3 from NUREG 8.39 (Ref. 8).

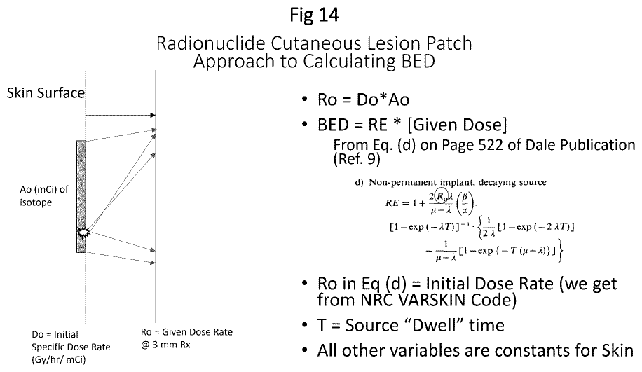

FIG. 14 is an illustration and mathematical example of the calculation of Biologically Effective Dose from the topical radiation patch treatment using a formula first defined in the British Journal of Radiology (Ref. 9).

### Comparison of the Device's Radiation Dose Delivery to Accepted Standards of Care

The radioactivity level of a device embodying features of the present invention should produce a dose and a BED in the patient that is reasonably close to the dose and BED produced by other types of radiotherapy devices and accepted treatment regimens. As a comparison to current standards of care, the dose and BED produced by the present invention may be compared to the most recent American Society for Radiation Oncology (ASTRO) Clinical Practice Guideline for skin cancer radiotherapy; see Ref. 11, Table 8. In the ASTRO table, the minimum BED of the accepted skin cancer treatment regimens is 58 Gy and the minimum dose delivered in a single fraction of a given regimen is 180 centiGray (cGy). Hence, a device embodying features of the present invention should provide a physical dose per fraction close to or exceeding this level of 180 cGy and a treatment regimen BED close to or exceeding this level of 58 Gy.

The total dose delivered by the radiation patch described herein is a function of (1) the “dwell time” or time that the patch spends affixed to the lesion beginning with the initial placement, and (2) the number of sequential treatments (or “fractions”), assuming all fractions have the same dwell time. The DAC curve in FIG. 13 may be used to determine the required activity per fraction to deliver a radiation dose (Gray) or Biologically Effective Dose (Gray-effective). As depicted in the DAC curve of FIG. 13, the maximum permissible activity is approximately 12 mCi in order for the patch to be considered releasable by NUREG 8.39 standards. Therefore as an example, to deliver a therapeutic dose of 60 Gray-effective (BED) the curve states that the number of fractions n=2 and the activity required per fraction is about 9.5 mCi. Hence, by using the DAC curve the number of required fractions, BED, activity in mCi and given dose may be determined for a variety of possible fractionation and dose delivery schemes.

FIG. 16 is an exploded view of one embodiment of the invention, which illustrates a sealed radiation delivery device 10 (the active layer depicted as 21, 23 and 25 in FIG. 1), a removable shield 50 which can be placed atop the sealed source that protects staff from unnecessary radiation exposure, and a bandage dressing 55 which may incorporate an alignment graticule 60 for accurate placement of the topical patch onto the diseased area. While the sealed radiation delivery device 10 containing the radioactive isotope 6, or if incorporated into a patch 8, should not be disassembled, the bandage dressing 55 and the removable radiation shield 50 are detachable from the sealed radiation delivery device 10. This is so that the staff can align the bandage 55 to the disease site first, and then place the sealed radiation delivery device 10 atop the alignment graticule 60. Finally, after the bandage dressing 55 has been applied and the sealed radiation delivery device 10 has been placed onto the alignment graticule 60, the removable shield 50 is placed over the sealed radiation delivery device 10 and disease site. The removable shield 50 then protecting the staff from unnecessary radiation exposure.

While there has been shown a preferred embodiment of the present invention, it is to be understood that certain changes may be made in the forms and arrangement of the elements, and the steps of the method, of the brachytherapy radiation delivery device without departing from the underlying spirit, scope, and essential characteristics of the invention. The present embodiment is therefore, to be considered as merely illustrative and not restrictive, the scope of the invention being indicated by the claims rather than the foregoing description, and all changes which come within the meaning and range of equivalence of the claims are therefore intended to be embraced therein.

What is claimed is:

1. A topical radiation delivery device, the device is designed to topically treat cutaneous skin lesions in patient tissue, the device comprising:
a central radioactive layer, the central radioactive layer comprising at least one uniformly distributed radionuclide and emitting radiation energy in all directions outward, the at least one radionuclide sealed within the radioactive layer;
a bolus bottom layer positioned below the central radioactive layer, and a radiation shielding top layer positioned above the central radioactive layer;
the central radioactive layer comprising at least one radionuclide emitting energy as gamma particles and beta particles; the at least one radionuclide in the central radioactive layer selected such that the combined energy of the emitted gamma and beta particles results in a desired Percent Depth Dose curve when the topical radiation delivery device is placed upon a cutaneous skin lesion, with the bolus bottom layer adjacent the cutaneous skin lesion, for a time period;
the molecular concentration of the at least one radionuclide in the central radioactive layer selected to cause the desired fluence of gamma and beta particles to be incident upon the skin lesion and the desired absorbed radiation dose to be deposited into the skin lesion over the time period; and
wherein the thickness of the bolus bottom layer is selected to attenuate the beta particle fluence at shallow skin depths and an optimal ratio of beta energy to gamma energy is deposited into the skin lesion to achieve a desired fraction of Biologically Effective Dose for the treatment of the cutaneous skin lesion.

2. The topical radiation delivery device of claim 1, wherein a at least one radionuclide emitting high energy gamma particles with energy greater than 1 MeV is selected to effectively counteract the shallowing effect of the inverse-square law and the Mayneord's effect at short source-to-target distances.

3. The topical radiation delivery device of claim 1, wherein when the radiation delivery device is placed upon a cutaneous skin lesion of a patent, the selected activity of a at least one radionuclide and the top layer radiation shielding ensure a member of the public cannot receive a dose of more than 5 milli-Sieverts.

4. The topical radiation delivery device of claim 1, wherein an additional removable portable radiation shield is affixed over the topical radiation delivery device, and ensures a member of the public and medical staff receive a dose that is as low as reasonably achievable.

5. The topical radiation delivery device of claim 1, wherein the at least one radionuclide comprises Na-24, La-140, Ga-66, Y-90, or any combination thereof.

6. The topical radiation delivery device of claim 1, wherein the at least one radionuclide comprises Na-24.

7. The topical radiation delivery device of claim 1, wherein the at least one radionuclide emits gamma-photons of energy between 0.5 MeV and 3.

8. The topical radiation delivery device of claim 1, wherein the at least one radionuclide comprises a gamma-emitter having a physical half-life between 1 hour and 72 hours.

9. The topical radiation delivery device of claim 1, wherein the topical radiation delivery device is topically affixed over the cutaneous skin lesion using a bandage dressing having a central holder for the radiation delivery device, for the duration of the prescribed time period.

10. A method to topically treat cutaneous skin lesions in patient tissue, the method comprising the steps of:
selecting at least one radionuclide, the at least one radionuclide emitting energy as gamma particles and beta particles;
calculating a required activity of the at least one radionuclide within the central radioactive layer to result in the desired absorbed radiation dose within the targeted lesion;
calculating a required bolus layer thickness to optimize the ratio of beta particle fluence relative to the total combined fluence of beta and gamma particle radiation energy resulting in an optimal Percentage Depth Dose curve within the patient's tissue;
preparing at least one radionuclide with the required radiation activity;
assembling a topical radiation delivery device, the topical radiation delivery device comprising; a shielding top layer, a bolus bottom layer, and a central radioactive layer;
the central radioactive layer comprising the at least one radionuclide uniformly distributed and emitting radiation energy in all directions outward, the at least one radionuclide sealed within the central radioactive layer;
the radiation device having the bolus bottom layer of the calculated thickness between the central radioactive layer and the cutaneous lesion;
positioning and securing the radiation device over the cutaneous skin lesion;
the topical radiation delivery device irradiating the cutaneous skin lesion over a time period, wherein a desired fraction of Biologically Effective Dose to the skin lesion is achieved; and
removing the topical radiation delivery device from the patient skin.

11. The method of claim 10, wherein a new topical radiation delivery device is applied over the cutaneous skin lesion to apply an additional fraction of the Biologically Effective Dose.

12. The step of claim 11, wherein the step applying a new topical radiation delivery device over the cutaneous skin lesion is repeated until the total Biologically Effective Dose is achieved.

13. The method of claim 10, wherein when the topical radiation delivery device is placed upon a cutaneous skin lesion of a patent, the device is substantially radiation sealed external to the patient by the radiation shielding top layer and the patients body.

14. The method of claim 10, wherein an additional removable portable radiation shield is affixed over the topical radiation delivery device after positioning and securing the radiation device over the cutaneous skin lesion and ensures a member of the public and medical staff receive a dose that is as low as reasonably achievable.

15. The method of claim 14, wherein when the topical radiation delivery device is placed upon a cutaneous skin lesion of a patent, the top layer radiation shielding and the additional removable portable radiation shield ensures a member of the public cannot receive a dose of more than 5 milli-Sieverts.

16. The method of claim 10, wherein the at least one radionuclide comprises Na-24, La-140, Ga-66, Y-90, or any combination thereof.

17. The method of claim 10, wherein the at least one radionuclide comprises Na-24.

18. The method of claim 10, wherein the at least one radionuclide emits gamma-photons of energy between 0.5 MeV and 3 MeV.

19. The method of claim 10, wherein the at least one radionuclide is a gamma-emitter having a physical half-life between 1 hour and 72 hours.

20. The method of claim 10, wherein the topical radiation delivery device is topically affixed over the cutaneous skin lesion using a bandage dressing having a central holder for the radiation delivery device, for the duration of the prescribed time period.
